(19) United States
(12) Patent Application Publication
(10) Pub. No.: US 2009/0264711 A1
(43) Pub. Date: Oct. 22, 2009

(54) BEHAVIOR MODIFICATION RECOMMENDER

(75) Inventors: Francesca Schuler, Des Plaines, IL (US); Krishna Jonnalagadda, Algonquin, IL (US)

Correspondence Address: MOTOROLA, INC., 1303 EAST ALGONQUIN ROAD, IL01/3RD, SCHAUMBURG, IL, 60196, US

(73) Assignee: MOTOROLA, INC., Schaumburg, IL (US)

(21) Appl. No.: 12/104,839

(22) Filed: Apr. 17, 2008

(51) Int. Cl. A61B 5/00; G08B 23/00

(52) U.S. Cl. 600/300; 340/573.1

(57) ABSTRACT

A method and apparatus for behavior modification is provided herein. During operation an apparatus (100) learns what context causes desired and undesired physiological parameters and then coaches a user to be aware of context causing undesired behavior and avoid future occurrences. The apparatus may then recommend behavior modification prior to the parameter reaching elevated status. Because an individual will be warned when situations exist that will cause an undesired physiological parameters, the apparatus will suggest to the user a method to alleviate the situation, for example, substituting an activity or context that creates desired physiological patterns. The user will then be able to take appropriate steps to alleviate the situation.

### FIELD OF THE INVENTION

The present invention relates generally to behavior modification and in particular, to a method and apparatus for recommending behavior modification.

### BACKGROUND OF THE INVENTION

Oftentimes individuals are instructed by their doctors to keep certain physiological parameters (heart rate, blood pressure, blood sugar level, respiratory rate, respiratory depth, joint wear, . . . , etc.) within certain boundaries. It is typically left to the individual to figure out the triggers that result in their elevated physiological parameters. It would be beneficial for such individuals if a system existed that would learn what triggered their elevated physiological parameter. Such a system could then provide feedback or coaching so that the individual could learn and modify their behavior prior to their physiological parameter reaching elevated status. Therefore a need exists for a method and apparatus for recommending behavior modification that identifies environmental context that cause both desired and undesired affects to physiological parameters, identifies patterns of these environmental triggers and recommends behavior modification or coaching to aid user in avoiding undesired behaviors in the future.

### BRIEF DESCRIPTION OF THE DRAWINGS

FIG. 1. is a block diagram of a recommender system.

FIG. 2. is a flow chart showing operation of the recommender system of FIG. 1.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION OF THE DRAWINGS

In order to alleviate the above-mentioned need, a method and apparatus for behavior modification is provided herein. During operation an apparatus learns what context causes desired and undesired physiological parameters and then coaches a user to be aware of context causing undesired behavior. The apparatus may then recommend behavior modification prior to the parameter reaching elevated status. Because an individual will be warned when situations exist that will cause an undesired physiological parameter, the apparatus will suggest to the user a method to alleviate the situation, for example, substituting an activity or context that creates desired physiological patterns. The user will then be able to take appropriate steps to alleviate the situation.

The present invention encompasses a method for facilitating behavior modification. The method comprises the steps of monitoring a user's physiological parameter, monitoring a user's context, deriving relationships between the user's physiological parameter and the user's context, and predicting either a desired or undesired physiological parameter based on the derived relationships.

The present invention additionally encompasses an apparatus comprising a biosensor monitoring a user's physiological parameter, context-aware circuitry monitoring a user's context, and logic circuitry deriving relationships between the user's physiological parameter and the user's context and predicting either a desired or undesired physiological parameter based on the derived relationships.

For clarity, the following description of the present invention will be given with stress level as the physiological parameter that will be monitored and controlled. Stress level is derived from measured physiological parameters such as heart rate and blood pressure. One of ordinary skill in the art will recognize, however, that other physiological parameters may be directly monitored and controlled in addition to stress. For example, blood pressure, heart rate, blood sugar level, respiratory depth, respiratory rate, joint wear, eye dryness, may be controlled by the following technique.

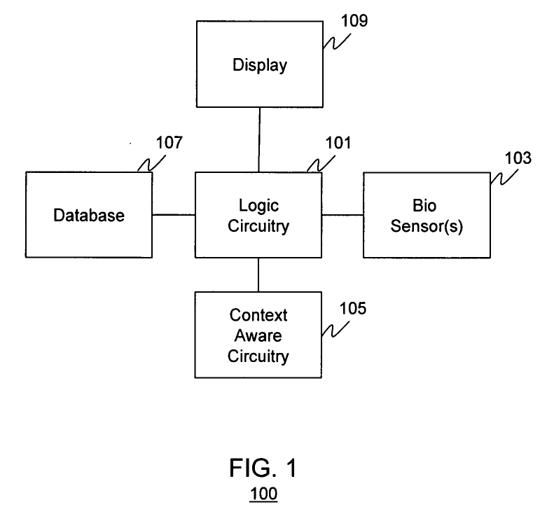

Turning now to the drawings, where like numerals designate like components, FIG. 1 is a block diagram showing apparatus 100 for behavior modification. As shown, apparatus 100 comprises logic circuitry 101, at least one biosensor 103 (a sensor where a physiological parameter can be directly or indirectly derived), context-aware circuitry 105, database 107, and display circuitry 109. Logic circuitry 101 comprises a digital signal processor (DSP), general purpose microprocessor, a programmable logic device, or application specific integrated circuit (ASIC) and is utilized to access and control apparatus 100. Logic circuitry 101 additionally uses biosensors 103 and context-aware circuitry 105 to determine those situations where an individual's stress level will be elevated as well as those situations where the individual's stress level is reduced.

Context-aware circuitry 105 may comprise any device capable of generating a current context. For example, context-aware circuitry 105 may comprise a GPS receiver capable of determining a location of the user device. Alternatively, circuitry 105 may comprise such things as a clock, calendar, environmental sensors (e.g. a thermometer capable of determining an ambient temperature, humidity, presence of dispersed chemicals, etc.), an accelerometer, a barometer, speech recognition circuitry, a user's electronic calendar, short-range communication circuitry (e.g., Bluetooth™ circuitry) to determine what other electronic devices are near . . . , etc.

Database 107 comprises standard random access memory and is used to store information related to undesired triggering events as well as desired events. Biosensors 103 comprise such things as a heart-rate monitor, an activity and motion detector (accelerometer), activity impact (pressure sensors), a blood-pressure monitor, respiratory sensors, blood sugar monitor, or a hydration sensor. Finally, display 109 comprises a standard liquid-crystal or other types of displays that is used to provide textual warnings and/or suggestions to the user. It should be noted that in an alternate embodiment a user can also get audio alert tones or coaching via text to speech engine via headset or a handset based speakerphone when appropriate.

As discussed above, it would be beneficial for individuals if a system existed that would learn what triggered their elevated stress and then provide some sort of feedback or coaching so that the individual could modify their behavior and recognize potential future occurrences. The system could also alert user to potential occurrences on the horizon. In order to provide for this need, apparatus 100 learns what context causes an elevated stress level for the user and then predicts when the user will have an elevated stress level. Apparatus 100 recommends behavior modification or coaching to aid user in avoiding undesired behaviors in the future or making them aware of the affect of the environmental context to their overall well being.

### Identifying Trigger Events:

Biosensors 103 may comprise a heart-rate monitor and/or a blood pressure monitor, while context-aware circuitry 105 comprises a GPS receiver, Bluetooth circuitry, and/or voice-recognition circuitry. During operation, logic circuitry will monitor biosensors 103 to determine stressful (high blood pressure, high heart rate) and non-stressful situations (lower blood pressure, lower heart rate). Stressful and non-stressful situations will be associated by logic circuitry 101 with various contextual situations.

It is a known fact that environmental factors such as location and activity cause stress: For example, location (in colleague's office), Activity (Driving in Traffic, Emailing at work), who a person is talking to (Boss), conversation topic, etc. can all lead to stressful situations. During operation, logic circuitry 101 retrieves context surrounding the condition of the heart rate (activity, communication channels, environment, etc.) and labels the condition as a desired or undesired behavior and determines if this condition is repeatable over time. If it is, logic circuitry 101 uses display 109 to coach the user as to what environmental context triggers stress, and may encourage the user to substitute stressful activities with non-stressful activities or avoid conditions and context causing a stressful activity.

As discussed above, apparatus 100 learns what contextual situations causes desired or undesired behavior patterns. In order to accomplish this, logic circuitry 101 monitors biosensors 103 over a period of time (e.g., several days) in order to determine situations when stress and non-stress occurs. For this particular embodiment, it is assumed that a person is under a stressful situation when their blood pressure and/or heart rate moves beyond a predetermined threshold for more than a predetermined period of time. For example, logic circuitry 101 may determine that a person is stressed when their blood pressure is above 140/90 (systolic pressure/diastolic pressure) for 5 minutes or their heart rate is above 110 for 5 minutes. However, if that elevated parameter is accompanied by extracting context that their physical activity intensity is high, this may not be considered a trigger event. If the activity level is low (extracted from motion, accelerometer, pressure sensors), then this is identified as a trigger event.

Once logic circuitry 101 determines that a stress situation exists, logic circuitry 101 will access context-aware circuitry 105 in order to analyze the contextual situations. For example, logic circuitry 101 may:
access an electronic calendar to determine if a person is in a particular meeting;
access location circuitry to determine a current location;
access a Bluetooth™ device in order to determine other devices in the vicinity, and hence infer other individuals who are in the vicinity;
access voice recognition circuitry in order to determine any key words from a conversation;
mine email or text message to determine key words from a discussion;
access motion sensors (e.g. pedometer, accelerometer, pressure sensors, etc.) to access the level and type of user activity;
access phone contact list to determine who the user communicated with (last phone number dialed is correlated to a contact list);
access a database to get additional location data (from a server—location type or label—restaurant, train station, etc.).

When logic circuitry 101 has identified stress triggers, and non-stress triggers, the triggers will be stored in database 107. Logic circuitry 101 will identify a particular context as a trigger when certain contextual information is similar for various stressful events. For example, logic circuitry 101 may determine that a stressful situation exists when the person is driving (as determined from context-aware circuitry 105 detecting an automobile's Bluetooth™ device is present and motion sensors identifying a moving automobile). This determination may be made due to the fact that several stressful events were identified when the user was in their automobile.

In a similar manner logic circuitry 101 may determine when non-stressful events occur. Non-stressful events may, for example, be inferred when the user's blood pressure is below 120/80 (systolic pressure/diastolic pressure) for 5 minutes or their heart rate is below 70 for 5 minutes. Logic circuitry 101 will identify a particular context as a non-stress trigger when certain contextual information is similar for various non-stressful events. For example, logic circuitry 101 may determine that a non-stressful situation exists when the person is talking on the phone to a particular individual. This determination may be made due to the fact that several non-stressful events were identified when the user was talking to this individual.

Interactions after Identification of Trigger and Non-Trigger Events:

After trigger and non-trigger events are identified, database 107 will contain these triggering events for desired and undesired behavior patterns. Logic circuitry 101 will then periodically monitor the user's context. If it is determined that a current context matches an undesired trigger, logic circuitry 101 may provide a visual or audio alert. A recommendation to substitute the current activity with an alternate one (e.g., a low-stress trigger) may be made.

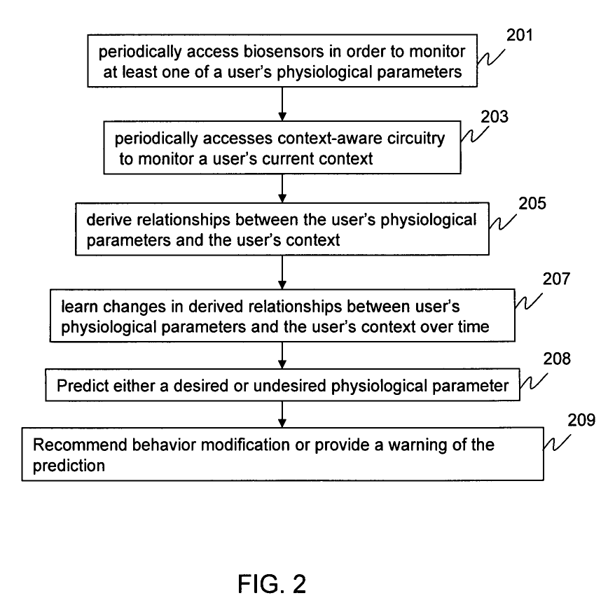

FIG. 2. is a flow chart showing operation of the recommender system when learning about a user. In particular, the steps of FIG. 2 show a method for facilitating behavior modification. The logic flow begins at step 201 where logic circuitry 101 periodically accesses biosensors 103 in order to monitor at least one of a user's physiological parameters (in this case heart rate and/or blood pressure). In embodiments of the present invention the biosensors may comprise an a heart-rate monitor, an activity and motion detector, an accelerometer, an activity impact sensor, a pressure sensor, a blood-pressure monitor, a respiratory sensor, a blood sugar monitor, or a hydration sensor.

Next, at step 203 logic circuitry periodically accesses context-aware circuitry 105 to monitor a user's current context. In this particular embodiment the current context comprises a user's location (determined via a GPS receiver), current activity, a user's current topic of conversation, who is in the user's vicinity (via Bluetooth circuitry), who the user is talking to (via Bluetooth and/or voice recognition circuitry), calendar information, applications used, and/or scheduled events.

At step 205 relationships are derived and learned between the user's physiological parameters (e.g., stress) and the user's context, where the derivation takes place over the course of several days and preferably across several locations. A relationship exists between the user's physiological parameter (e.g., stress) and the user's context when it has been determined that the context causes a predictable outcome in the user's physiological parameter (stress level). During step 205, logic circuitry 101 may access database 107 to associate particular geographic locations with a place of business. For example, database 107 may comprise a local listing of businesses along with their locations. Instead of simply identifying a relationship between geographic coordinates and stress, logic circuitry 101 may access database 107 to find the business that causes the stress (e.g., a shopping center) and then make an association between that business and stress. The derived relationships are not static over time, but dynamic. During step 207, logic circuitry 101 monitors changes in desired and undesired patterns over time. For example, context that once provoked an elevated parameter, may no longer provoke the elevated parameter.

Once the relationships are derived and learned, the logic flow continues to step 208 where logic circuitry monitors the user's context and predicts either a desired or undesired physiological parameters (e.g. stress) based on the derived and learned relationships and accesses logic circuitry used to display 109 to provide the user with a warning of the prediction. At step 209, logic circuitry may also use display 109 to coach the user on alternative behaviors/activities when stressful situations occur, or may occur. As discussed above, the alternative behaviors/activities may be predicted to cause a desired physiological parameter in a similar way that certain behaviors cause an undesired physiological parameter.

While the invention has been particularly shown and described with reference to a particular embodiment, it will be understood by those skilled in the art that various changes in form and details may be made therein without departing from the spirit and scope of the invention. For example, while the above description was given with regards to monitoring stress, other physiological parameters may be monitored and predicted in a similar manner. For example:
Blood sugar level rises or drops based on level of activity, and therefore is not necessarily dependent solely on food intake. Apparatus 100 could monitor activity level as it relates to blood sugar (via accelerometer, pedometer, pressure sensors) and make recommendations as to the level of activity to maintain appropriate glucose levels or when food is required if activity continues. For example, apparatus 100 can coach user to avoid high levels of activity without appropriate food intake.
Joint wear is dependent on surface type, level of activity, shoe type, and incline. Increased impact to joints can be detected via pressure sensors on shoes. Logic circuitry 101 can retrieve context causing increased impact (i.e. detect activity via accelerometer, location of trail and incline/decline via GPS, and via a database capture the surface type and coaches users to avoid activity (running, jogging) at certain locations, certain inclines, certain surface types, or make substitutes for low impact activities or alert user to shoe wear, etc.
Accelerometer and motion sensors in addition to pressure sensors can indicate issues with a person's gait or posture. There may be certain activities or activity types or other context that may cause a person to have a poor gait or posture. Logic circuitry 100 can detect this context and provide coaching to substitute other activities that have a positive or less negative impact on a person's gait and posture.
   It is intended that such changes come within the scope of the following claims:

1. A method for facilitating behavior modification, the method comprising the steps of:
monitoring a user's physiological parameter;
monitoring a user's context;
deriving relationships between the user's physiological parameter and the user's context, wherein the derivation takes place over the course of several days across several locations;
learning of changes in derived relationships over time; and
predicting either a desired or undesired physiological parameter based on the derived and learned changes in derived relationships.

2. The method of claim 1 wherein the context comprises at least one of the following:
the user's location,
the user's current topic of conversation,
who is in the user's vicinity,
who the user is talking to,
calendar information,
scheduled events, or
applications used.

3. The method of claim 1 further comprising the step of:
providing the user a warning of the prediction.

4. The method of claim 1 wherein the step of monitoring the user's physiological parameters comprises the step of monitoring with a biosensor.

5. The method of claim 4 wherein the biosensor comprises a biosensor taken from the group consisting of a heart-rate monitor, an activity and motion detector, an accelerometer, an activity impact sensor, a pressure sensor, a blood-pressure monitor, a respiratory sensor, a blood sugar monitor, and a hydration sensor.

6. The method of claim 1 wherein the step of monitoring the user's context comprises the step of monitoring the user's context with a GPS receiver, short range communication circuitry including Bluetooth, or voice-recognition circuitry.

7. The method of claim 6 wherein the step of monitoring a user's context further comprises accessing a database to associate a location with a place of business.

8. The method of claim 1 wherein a relationship exists between the user's physiological parameter and the user's context when it has been determined that the context causes a predictable outcome in the user's physiological parameter.

9. The method of claim 1 further comprising the step of:
providing the user an alternative activity when an undesired physiological parameter is predicted.

10. The method of claim 9 wherein the alternative activity is found to cause a predictable desired physiological parameter.

11. The method of claim 1 wherein the physiological parameter comprises stress.

12. The method of claim 1 wherein the physiological parameter comprises blood sugar level, joint wear, or a person's gait or posture.

13. The method of claim 1 further comprising the step of:
learning of changes in derived relationships over time; and
predicting either a desired or undesired physiological parameter based on the derived and learned relationships.

14. An apparatus comprising:
a biosensor monitoring a user's physiological parameter;
context-aware circuitry monitoring a user's context; and
logic circuitry deriving relationships between the user's physiological parameter and the user's context and predicting either a desired or undesired physiological parameter based on the derived relationships, wherein the derivation takes place over the course of several days across several locations.

15. The apparatus of claim 14 wherein the context comprises at least one of the following:
the user's location,
the user's current topic of conversation,
who is in the user's vicinity,
who the user is talking to,
calendar information,
scheduled events, or
applications used.

16. The apparatus of claim 14 further comprising:
display circuitry providing the user a warning of the prediction.

17. The apparatus of claim 14 wherein the biosensor comprises a biosensor taken from the group consisting of a heart-rate monitor, an activity and motion detector, an accelerometer, an activity impact sensor, a pressure sensor, a blood-pressure monitor, a respiratory sensor, a blood sugar monitor, and a hydration sensor.

18. The apparatus of claim 14 wherein the context-aware circuitry comprises a GPS receiver, Bluetooth circuitry, or voice-recognition circuitry.

19. The apparatus of claim 14 further comprising a database associating a location with a place of business.

20. The apparatus of claim 14 wherein a relationship exists between the user's physiological parameter and the user's context when it has been determined that the context causes a predictable outcome in the user's physiological parameter.
